(19) United States
(12) Patent Application Publication
(10) Pub. No.: US 2024/0042174 A1
(43) Pub. Date: Feb. 8, 2024

(54) INTRAVASCULAR BENDING OF MEDICAL INSTRUMENTS

(71) Applicant: SV SWISSVORTEX AG, Zurich (CH)

(72) Inventors: Andrea GUIDOTTI, Zurich (CH); Pietro GOZZOLI, Zurich (CH); Idan TOBIS, Beth Hashmonai (IL)

(73) Assignee: SV SWISSVORTEX AG, Zurich (CH)

(21) Appl. No.: 18/266,492

(22) PCT Filed: Dec. 9, 2021
(86) PCT No.: PCT/IB2021/061477

§ 371 Date: Jun. 9, 2023

Related U.S. Application Data
(60) Provisional application No. 63/123,534, filed on Dec. 10, 2020.
(60) Provisional application No. 63/123,533, filed on Dec. 10, 2020.

(51) Int. Cl. A61M 25/01

(52) U.S. Cl. CPC A61M 25/0147; A61M 2025/0163; A61M 2025/015

(57) ABSTRACT

A probe (24) is provided for use with an elongate medical instrument (20). The probe (24) is configured to be advanced through vasculature of a body of a subject, and includes a distal longitudinal probe-portion (26) and one or more loops (30). The one or more loops (30) are disposed along the distal longitudinal probe-portion (26) and are configured to be looped around a distal longitudinal medical-instrument-portion (28) of the elongate medical instrument (20) when the distal longitudinal probe-portion (26) is positioned at least partially alongside a distal longitudinal medical-instrument-portion (28) within the body, thereby mechanically coupling the distal longitudinal probe-portion (26) to the distal longitudinal medical-instrument-portion (28) within the body. The probe (24) is configured to bend the elongate medical instrument (20) within the body when the distal longitudinal probe-portion (26) is mechanically coupled to the distal longitudinal medical-instrument-portion (28). Other embodiments are also described.

### CROSS-REFERENCE TO RELATED APPLICATIONS

The present patent application claims priority from U.S. Provisional Application 63/123,533, filed Dec. 10, 2020, and U.S. Provisional Application 63/123,534, filed Dec. 10, 2020, which are assigned to the assignee of the present application and incorporated herein by reference.

### FIELD OF THE INVENTION

The present invention relates generally to medical deployment tools and methods, and more particularly to intravascular deployment tools and methods.

### BACKGROUND OF THE INVENTION

Modern cardiovascular therapies include the use of positioning catheters to navigate the intended therapy to a targeted anatomical location. The trajectory of these catheters requires steerability features that are usually integrated within the shaft body of the catheter, in order to achieve different degrees and planes of steering.

Currently available steerable percutaneous catheters and delivery systems require considerable diameters to allow steerability.

### SUMMARY OF THE INVENTION

Some embodiments of the present invention provide a probe and a method of using the probe to intravascularly bend an elongate medical instrument, such as an intracardiac medical instrument, a brain medical instrument, a diagnostic medical instrument, and/or a therapeutic medical instrument. The method comprises advancing the elongate medical instrument through vasculature, advancing the probe through the vasculature, and positioning a distal longitudinal probe-portion of the probe at least partially alongside a distal longitudinal medical-instrument-portion of the elongate medical instrument. Thereafter, the distal longitudinal probe-portion is mechanically coupled to the distal longitudinal medical-instrument-portion, and the probe is used to bend the elongate medical instrument. The probe is configured to bend the elongate medical instrument within the body when the distal longitudinal probe-portion is mechanically coupled to the distal longitudinal medical-instrument-portion.

The probe and this method provide steerability to any the elongate medical instrument that lacks a steerability-providing mechanism, or provide additional steerability to any the elongate medical instrument that has a steerability-providing mechanism, such as to in order to provide additional directionality and/or a smaller bend radius to the existing steerability-providing mechanism. An alternative approach of integrating steerability functionality, or additional steerability functionality, directly into the elongate medical instrument during manufacture thereof would require adding additional channels to the elongate medical instrument to accommodate steering wire(s), which might increase the diameter of the elongate medical instrument. The reduced diameter of the elongate medical instrument provided by the probe may enhance minimally invasive procedures, such as by reducing the risk of complications. In addition, the probe is only utilized in cases in which steerability is required, which may provide procedural cost savings. Moreover, in some challenging anatomies, elongate medical instruments that usually do not integrate or require steering features may benefit from the external steerability aid provided by the probe.

In addition, since the probe is coupled to the elongate medical instrument in situ within the vasculature, the additional steerability is provided to the elongate medical instrument without the need for a larger diameter delivery catheter for the elongate medical instrument than would otherwise be needed.

The probe and this method also allow the healthcare worker to configure the resulting steering properties (e.g., bending point) of the elongate medical instrument, such as by selecting the one or more locations along the elongate medical instrument at which the distal longitudinal probe-portion is coupled to the distal longitudinal medical-instrument-portion. For example, steering properties of the elongate medical instrument may be configured based on the relative flexibility of different longitudinal segments of the elongate medical instrument. Optionally, the healthcare worker may configure the steering properties based on medical imaging (e.g., a CT scan) and/or a planned vascular path of the particular procedure and the particular patient's anatomy.

For some applications, the elongate medical instrument is advanced through the vasculature along a first route through the vasculature, and the probe is advanced through the vasculature along a second route through the vasculature, the second route different from the first route.

For some applications, the distal longitudinal probe-portion comprises one or more loops, which are configured to be looped around the distal longitudinal medical-instrument-portion when the distal longitudinal probe-portion is positioned at least partially alongside the distal longitudinal medical-instrument-portion within the body, thereby mechanically coupling the distal longitudinal probe-portion to the distal longitudinal medical-instrument-portion within the body. The distal longitudinal probe-portion is mechanically coupled to the distal longitudinal medical-instrument-portion by looping the one or more loops around the distal longitudinal medical-instrument-portion.

Some embodiments of the present invention provide an attachable bending system and a method of using the attachable bending system to intravascularly bend the elongate medical instrument, in accordance with some applications of the present invention.

The attachable bending system comprises:
a distal-most connector, which is configured to be mechanically coupled to the elongate medical instrument at a distal location along the elongate medical instrument;
one or more proximal-more connectors, which are configured to be mechanically coupled to the elongate medical instrument at one or more proximal-more locations along the elongate medical instrument, respectively, the one or more proximal-more locations more proximal than the distal location; and
a wire, which is fixed to the distal-most connector and slidably coupled to the one or more proximal-more connectors.

The distal-most connector, the one or more proximal-more connectors, and the wire are arranged such that proximal pulling of the wire bends the elongate medical instrument within the body of the subject when the distal-most connector and the one or more proximal-more connectors are coupled to the elongate medical instrument and the elongate medical instrument is within vasculature of the body.

The method of using the attachable bending system comprises:
mechanically coupling the distal-most connector to the elongate medical instrument at a distal location along the elongate medical instrument;
mechanically coupling the one or more proximal-more connectors to the elongate medical instrument at the one or more proximal-more locations along the elongate medical instrument, respectively, the one or more proximal-more locations 142 more proximal than the distal location;
thereafter, introducing the elongate medical instrument into the vasculature and advancing the elongate medical instrument through the vasculature; and
thereafter, bending the elongate medical instrument by pulling proximally on the wire fixed to the distal-most connector and slidably coupled to the one or more proximal-more connectors.

The attachable bending system and this method provide steerability to any the elongate medical instrument that lacks a steerability-providing mechanism, or provides additional steerability to any the elongate medical instrument that has a steerability-providing mechanism, such as to in order to provide additional directionality and/or a smaller bend radius to the existing steerability-providing mechanism. An alternative approach of integrating steerability functionality, or additional steerability functionality, directly into the elongate medical instrument during manufacture thereof would require adding additional channels to the elongate medical instrument to accommodate steering wire(s), which might increase the diameter of the elongate medical instrument.

In addition, since the distal-most connector and the one or more proximal-more connectors are coupled to the elongate medical instrument before the elongate medical instrument is introduced into the vasculature, practicing the method does not require additional vascular access than would otherwise be needed.

The attachable bending system and this method also allow the healthcare worker to configure the resulting steering properties (e.g., bending point) of the elongate medical instrument, such as by selecting the distal location and the one or more proximal-more locations along the elongate medical instrument at which the distal-most connector and the one or more proximal-more connectors, respectively, are coupled to the elongate medical instrument. For example, steering properties of the elongate medical instrument may be configured based on the relative flexibility of different longitudinal segments of the elongate medical instrument. Optionally, the healthcare worker may configure the steering properties based on medical imaging (e.g., a CT scan) and/or a planned vascular path of the particular procedure and the particular patient's anatomy.

For some applications, the one or more proximal-more connectors are shaped so as define one or more respective channels therethrough, and the wire passes through the one or more channels.

There is therefore provided, in accordance with an application of the present invention, a probe for use with an elongate medical instrument, the probe configured to be advanced through vasculature of a body of a subject, the probe including:
a distal longitudinal probe-portion; and
one or more loops, which are disposed along the distal longitudinal probe-portion, and which are configured to be looped around a distal longitudinal medical-instrument-portion of the elongate medical instrument when the distal longitudinal probe-portion is positioned at least partially alongside a distal longitudinal medical-instrument-portion within the body, thereby mechanically coupling the distal longitudinal probe-portion to the distal longitudinal medical-instrument-portion within the body,
wherein the probe is configured to bend the elongate medical instrument within the body when the distal longitudinal probe-portion is mechanically coupled to the distal longitudinal medical-instrument-portion.

For some applications, the one or more loops are configured to be looped around the distal longitudinal medical-instrument-portion by passing the one or more loops over the distal end of the distal longitudinal medical-instrument-portion, and thereafter proximally withdrawing the distal longitudinal probe-portion or distally advancing the distal longitudinal medical-instrument-portion, such that the one or more loops are positioned around the distal longitudinal medical-instrument-portion.

For some applications, the distal longitudinal probe-portion includes two or more loops, which are configured to be looped around the distal longitudinal medical-instrument-portion.

For some applications, the one or more loops are configured to be tightened around the distal longitudinal medical-instrument-portion after being looped around the distal longitudinal medical-instrument-portion, so as to mechanically couple the distal longitudinal probe-portion to the distal longitudinal medical-instrument-portion.

For some applications, the probe further includes one or more control wires that pass along the probe, and the one or more loops extend from one or more distal portions of the one or more control wires, and are arranged such that proximal pulling of the one or more control wires tightens the one or more loops around the distal longitudinal medical-instrument-portion.

For some applications, the one or more loops are configured to assume a non-radially-extended configuration and a radially-extended configuration.

For some applications, the one or more loops are retracted into the probe when in the non-radially-extended configuration.

For some applications, the one or more loops are retracted against the probe when in the non-radially-extended configuration.

For some applications, the probe further includes one or more control wires that pass along the probe and are configured to control a transition between the non-radially-extended configuration and the radially-extended configuration.

For some applications, the one or more loops are pivotable with respect to an external surface of the probe, so as to transition between the non-radially-extended configuration and the radially-extended configuration.

For some applications, the probe is axially non-compressible.

For some applications, the probe further includes a steerability-providing mechanism, which is configured to steer the probe so as to bend the elongate medical instrument within the body when the distal longitudinal probe-portion is mechanically coupled to the distal longitudinal medical-instrument-portion.

For some applications, the steerability-providing mechanism includes one or more wires that pass through one or more channels defined by a wall of the probe, and the steerability-providing mechanism is configured such that proximal pulling of at least one of the one or more wires bends the probe.

For some applications, the one or more wires are attached to the one or more loops.

For some applications, the one or more wires are integral with the one or more loops.

For some applications, a kit including the probe is provided, the kit further including the elongate medical instrument.

For some applications, the elongate medical instrument includes an intracardiac medical instrument, which is configured to be advanced through the vasculature into a heart chamber of the subject.

For some applications, the elongate medical instrument includes a brain medical instrument, which is configured to be advanced through the vasculature into a brain of the subject.

For some applications, the elongate medical instrument does not include a steerability-providing mechanism.

For some applications, the elongate medical instrument is shaped so as to define at least one longitudinal channel therethrough.

For some applications, the elongate medical instrument is not shaped so as to define any longitudinal channels therethrough.

For some applications, the elongate medical instrument includes a diagnostic medical instrument.

For some applications, the diagnostic medical instrument is selected from the group consisting of: an intracardiac echocardiography (ICE) probe, a biopsy probe, and an ultrasound probe.

For some applications, the elongate medical instrument includes a therapeutic medical instrument.

For some applications, the therapeutic medical instrument includes an ablation probe.

There is further provided, in accordance with an application of the present invention, an attachable bending system for use with an elongate medical instrument, the attachable bending system including:
a distal-most connector, which is configured to be mechanically coupled to the elongate medical instrument at a distal location along the elongate medical instrument;
one or more proximal-more connectors, which are configured to be mechanically coupled to the elongate medical instrument at one or more proximal-more locations along the elongate medical instrument, respectively, the one or more proximal-more locations more proximal than the distal location; and
a wire, which is fixed to the distal-most connector and slidably coupled to the one or more proximal-more connectors,
wherein the distal-most connector, the one or more proximal-more connectors, and the wire are arranged such that proximal pulling of the wire bends the elongate medical instrument within a body of a subject when the distal-most connector and the one or more proximal-more connectors are coupled to the elongate medical instrument and the elongate medical instrument is within vasculature of the body.

For some applications, the one or more proximal-more connectors are shaped so as define one or more respective channels therethrough, and the wire passes through the one or more channels.

For some applications, the distal-most connector includes a loop, and the distal-most connector is configured to mechanically coupled to the elongate medical instrument by looping the loop around the elongate medical instrument.

For some applications, a kit including the attachable bending system is provided, the kit further including the elongate medical instrument.

For some applications, the elongate medical instrument includes an intracardiac medical instrument, which is configured to be advanced through the vasculature into a heart chamber of the subject.

For some applications, the elongate medical instrument includes a brain medical instrument, which is configured to be advanced through the vasculature into a brain of the subject.

For some applications, the elongate medical instrument does not include a steerability-providing mechanism.

For some applications, the elongate medical instrument is shaped so as to define at least one longitudinal channel therethrough.

For some applications, the elongate medical instrument is not shaped so as to define any longitudinal channels therethrough.

For some applications, the elongate medical instrument includes a diagnostic medical instrument.

For some applications, the diagnostic medical instrument is selected from the group consisting of: an intracardiac echocardiography (ICE) probe, a biopsy probe, and an ultrasound probe.

For some applications, the elongate medical instrument includes a therapeutic medical instrument.

For some applications, the therapeutic medical instrument includes an ablation probe.

There is still further provided, in accordance with an application of the present invention, a method including:
advancing an elongate medical instrument through vasculature of a body of a subject;
advancing a probe through the vasculature and positioning a distal longitudinal probe-portion of the probe at least partially alongside a distal longitudinal medical-instrument-portion of the elongate medical instrument;
thereafter, mechanically coupling the distal longitudinal probe-portion to the distal longitudinal medical-instrument-portion within the body; and
thereafter, using the probe to bend the elongate medical instrument within the body.

For some applications, the distal longitudinal probe-portion includes one or more loops, and mechanically coupling includes looping the one or more loops around the distal longitudinal medical-instrument-portion.

For some applications, looping the one or more loops around the distal longitudinal medical-instrument-portion includes:
passing the one or more loops over the distal end of the distal longitudinal medical-instrument-portion; and
thereafter, proximally withdrawing the distal longitudinal probe-portion or distally advancing the distal longitudinal medical-instrument-portion, such that the one or more loops are positioned around the distal longitudinal medical-instrument-portion.

For some applications, the distal longitudinal probe-portion includes two or more loops, and mechanically coupling includes looping the two or more loops around the distal longitudinal medical-instrument-portion.

For some applications, mechanically coupling the distal longitudinal probe-portion to the distal longitudinal medical-instrument-portion includes tightening the one or more loops around the one or more loops around the distal longitudinal medical-instrument-portion after looping the one or more loops around the distal longitudinal medical-instrument-portion.

For some applications:
advancing the probe through the vasculature includes advancing the probe through the vasculature while the one or more loops are in a non-radially-extended configuration, and
mechanically coupling the distal longitudinal probe-portion to the distal longitudinal medical-instrument-portion includes transitioning the one or more loops to a radially-extended configuration.

For some applications, the one or more loops are retracted into the probe when in the non-radially-extended configuration.

For some applications, the one or more loops are retracted against the probe when in the non-radially-extended configuration.

For some applications, transitioning the one or more loops to the radially-extended configuration includes using one or more control wires that pass along the probe to transition the one or more loops to the radially-extended configuration.

For some applications, transitioning the one or more loops to the radially-extended configuration includes pivoting the one or more loops with respect to an external surface of the probe.

For some applications, the probe is axially non-compressible.

For some applications:
advancing the elongate medical instrument through the vasculature includes advancing the elongate medical instrument through the vasculature along a first route through the vasculature, and
advancing the probe through the vasculature includes advancing the probe through the vasculature along a second route through the vasculature, the second route different from the first route.

For some applications, the probe includes a steerability-providing mechanism, and using the probe to bend the elongate medical instrument includes activating the steerability-providing mechanism to steer the probe.

For some applications, the steerability-providing mechanism includes one or more wires that pass through one or more channels defined by a wall of the probe, and activating the steerability-providing mechanism includes pulling at least one of the one or more wires proximally to bend the probe.

For some applications:
the distal longitudinal probe-portion includes one or more loops,
the one or more wires are attached to or integral with the one or more loops, and
mechanically coupling includes looping the one or more loops around the distal longitudinal medical-instrument-portion.

For some applications, the elongate medical instrument is an intracardiac medical instrument, and advancing the elongate medical instrument includes advancing the intracardiac medical instrument through the vasculature into a heart chamber of the subject.

For some applications, the elongate medical instrument is a brain medical instrument, and advancing the elongate medical instrument includes advancing the brain medical instrument through the vasculature into a brain of the subject.

For some applications, the elongate medical instrument does not include a steerability-providing mechanism.

For some applications, the elongate medical instrument is shaped so as to define at least one longitudinal channel therethrough.

For some applications, the elongate medical instrument is not shaped so as to define any longitudinal channels therethrough.

For some applications, the elongate medical instrument is a diagnostic medical instrument.

For some applications, the diagnostic medical instrument is selected from the group consisting of: an intracardiac echocardiography (ICE) probe, a biopsy probe, and an ultrasound probe.

For some applications, the elongate medical instrument is a therapeutic medical instrument.

For some applications, the therapeutic medical instrument is an ablation probe.

There is additionally provided, in accordance with an application of the present invention, a method including:
mechanically coupling a distal-most connector to an elongate medical instrument at a distal location along the elongate medical instrument;
mechanically coupling one or more proximal-more connectors to the elongate medical instrument at one or more proximal-more locations along the elongate medical instrument, respectively, the one or more proximal-more locations more proximal than the distal location;
thereafter, introducing the elongate medical instrument into vasculature of a body of a subject and advancing the elongate medical instrument through the vasculature; and
thereafter, bending the elongate medical instrument within the body by pulling proximally on a wire fixed to the distal-most connector and slidably coupled to the one or more proximal-more connectors.

For some applications, the one or more proximal-more connectors are shaped so as define one or more respective channels therethrough, and the wire passes through the one or more channels.

For some applications, mechanically coupling the distal-most connector and the one or more proximal-more connectors to the elongate medical instrument includes gluing the distal-most connector and the one or more proximal-more connectors to the elongate medical instrument.

For some applications, the distal-most connector includes a loop, and mechanically coupling the distal-most connector to the elongate medical instrument includes looping the loop around the elongate medical instrument.

For some applications, the elongate medical instrument is an intracardiac medical instrument, and advancing the elongate medical instrument includes advancing the intracardiac medical instrument through the vasculature into a heart chamber of the subject.

For some applications, the elongate medical instrument is a brain medical instrument, and advancing the elongate medical instrument includes advancing the brain medical instrument through the vasculature into a brain of the subject.

For some applications, the elongate medical instrument does not include a steerability-providing mechanism.

For some applications, the elongate medical instrument is shaped so as to define at least one longitudinal channel therethrough.

For some applications, the elongate medical instrument is not shaped so as to define any longitudinal channels therethrough.

For some applications, the elongate medical instrument is a diagnostic medical instrument.

For some applications, the diagnostic medical instrument is selected from the group consisting of: an intracardiac echocardiography (ICE) probe, a biopsy probe, and an ultrasound probe.

For some applications, the elongate medical instrument is a therapeutic medical instrument.

For some applications, the therapeutic medical instrument is an ablation probe.

The present invention will be more fully understood from the following detailed description of embodiments thereof, taken together with the drawings, in which:

### BRIEF DESCRIPTION OF THE DRAWINGS

FIGS. 1A-B are schematic illustrations of a probe and a method of using the probe for intravascularly bending an elongate medical instrument, in accordance with some applications of the present invention;

FIGS. 2A-E are schematic illustrations of the probe and the method of FIGS. 1A-B, in accordance with some applications of the present invention;

FIG. 3 is a schematic illustration of another configuration of a probe used to perform the method of FIGS. 1A-B and 2A-E, in accordance with an application of the present invention;

FIGS. 4A-B are schematic illustrations of yet another configuration of a probe used to perform the method of FIGS. 1A-B and 2A-E, in accordance with an application of the present invention;

FIGS. 5A-B are schematic illustrations of still another configuration of a probe used to perform the method of FIGS. 1A-B and 2A-E, in accordance with an application of the present invention;

FIGS. 6A-B are schematic illustrations of another configuration of a probe used to perform the method of FIGS. 1A-B and 2A-E, in accordance with an application of the present invention;

FIGS. 7A-B are schematic illustrations of additional configurations of a probe used to perform the method of FIGS. 1A-B and 2A-E, in accordance with respective applications of the present invention;

FIGS. 8A-C are schematic illustrations of a configuration of a probe comprising a steerability-providing mechanism, in accordance with an application of the present invention;

FIGS. 9A-E are schematic illustrations of another configuration of a probe 24 comprising a steerability-providing mechanism, in accordance with an application of the present invention;

FIGS. 10A-B are schematic illustrations of a configuration of a probe comprising an alternative steerability-providing mechanism, in accordance with an application of the present invention;

FIGS. 11A-B are schematic illustrations of an attachable bending system and a method for intravascularly bending an elongate medical instrument using the attachable bending system, in accordance with some applications of the present invention; and

FIG. 12 is a schematic illustration of a portion of the attachable bending system of FIGS. 11A-B, in accordance with some applications of the present invention.

### DETAILED DESCRIPTION OF EMBODIMENTS

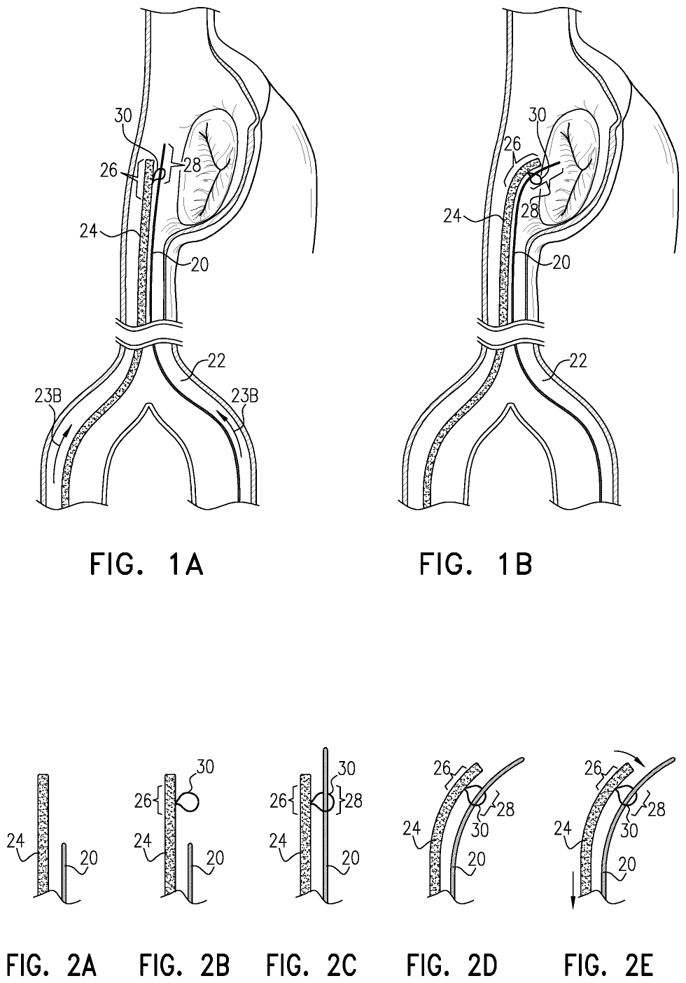

Reference is made to FIGS. 1A-B, which are schematic illustrations of a probe 24 and a method of using probe 24 to intravascularly bend an elongate medical instrument 20, in accordance with some applications of the present invention. Reference is also made FIGS. 2A-E, which are schematic illustrations of the method of FIGS. 1A-B, in accordance with some applications of the present invention. FIGS. 1A-B show vasculature 22 of a subject, while FIGS. 2A-E do not show the vasculature.

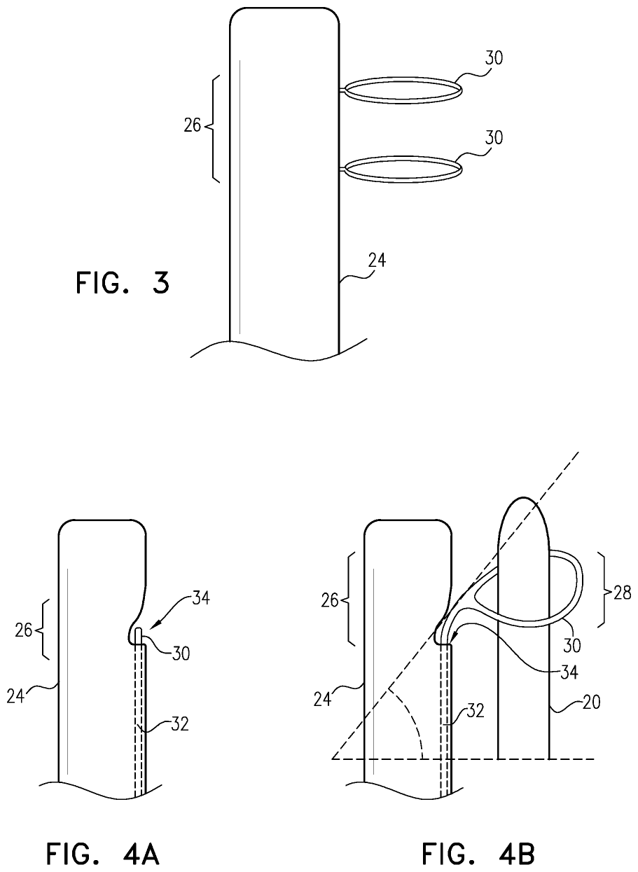

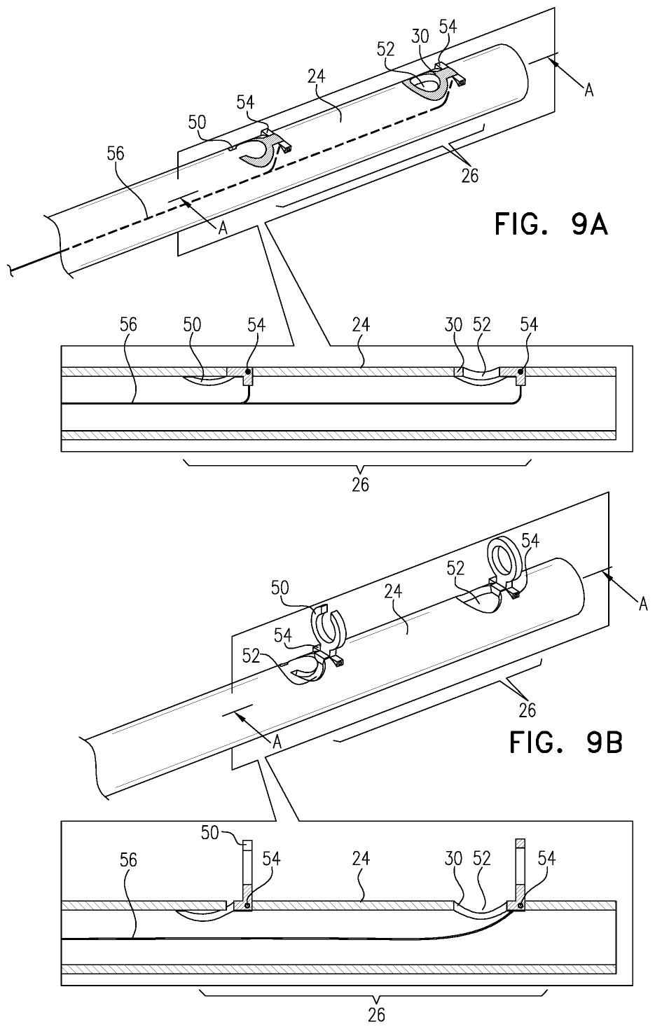

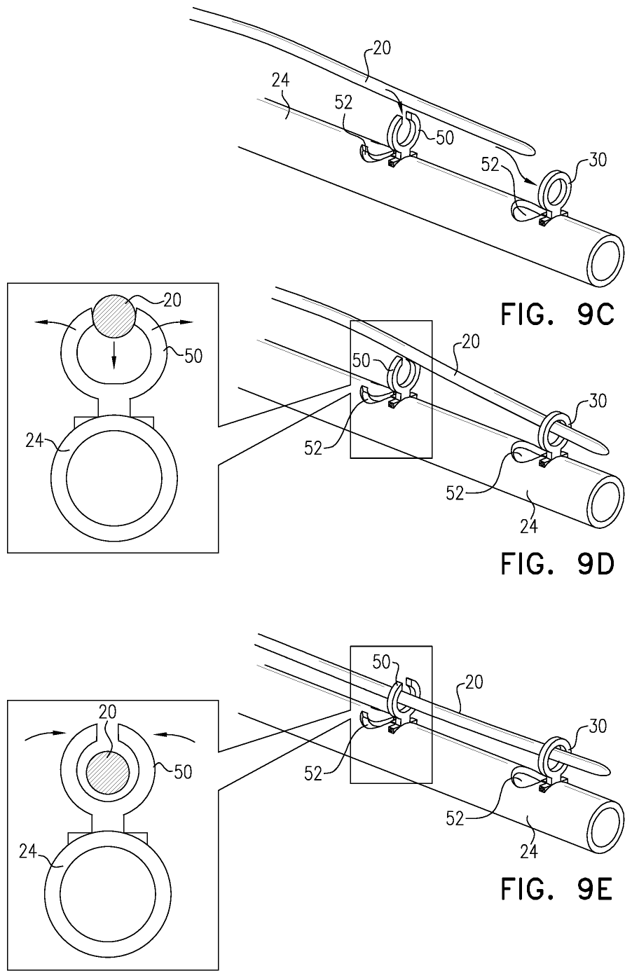

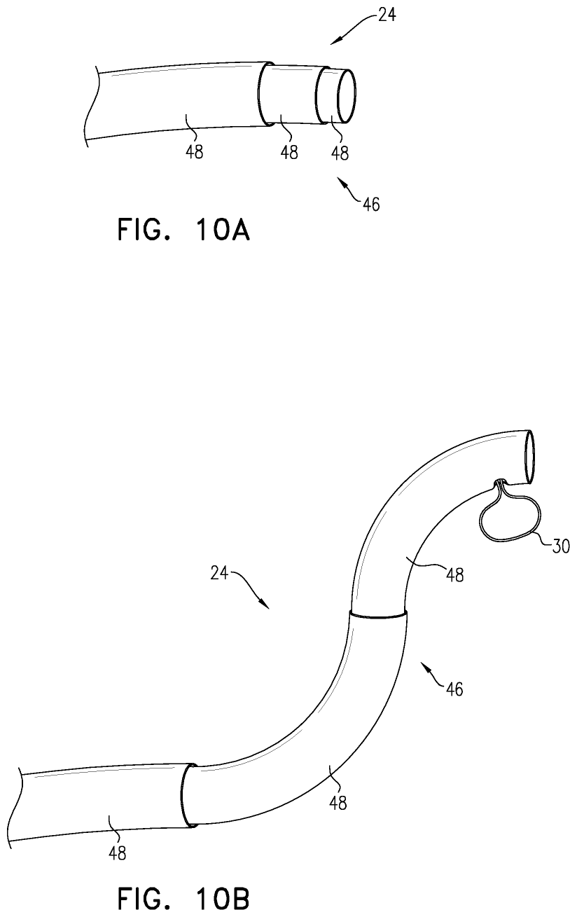

The method comprises:
advancing elongate medical instrument 20 through vasculature 22 of a subject (optionally, the advancing is performed when one or more loops 30 are in a non-radially-extended configuration, e.g., retracted into and/or against probe 24, such as shown in FIG. 2A and described hereinbelow with reference to FIGS. 4A, 5A, 6A, and 10A; or in a collapsed configuration, such as shown in FIG. 9A, described hereinbelow);
advancing probe 24 through vasculature 22 and positioning a distal longitudinal probe-portion 26 of probe 24 at least partially alongside a distal longitudinal medical-instrument-portion 28 of elongate medical instrument 20, such as shown in FIG. 2B;
thereafter, mechanically coupling distal longitudinal probe-portion 26 to distal longitudinal medical-instrument-portion 28, such as shown in FIGS. 1A and 2C (optionally, the mechanically coupling is performed after one or more loops 30 are transitioned to a radially-extended configuration in which the one or more loops 30 are extended away from probe 24, such as shown in FIGS. 1A and 2B, and described hereinbelow with reference to FIGS. 3, 4B, 5B, 6B, 7A-B, 8A-C, 9B-E, and 10B); and
thereafter, using probe 24 to bend elongate medical instrument 20, such as shown in FIGS. 1B and 2D-E.

Probe 24 is configured to bend elongate medical instrument 20 within the body when distal longitudinal probe-portion 26 is mechanically coupled to distal longitudinal medical-instrument-portion 28.

Probe 24 and this method provide steerability to any elongate medical instrument that lacks a steerability-providing mechanism, or provide additional steerability to any elongate medical instrument 20 that has a steerability-providing mechanism, such as to in order to provide additional directionality and/or a smaller bend radius to the existing steerability-providing mechanism. An alternative approach of integrating steerability functionality, or additional steerability functionality, directly into the elongate medical instrument during manufacture thereof would require adding additional channels to the elongate medical instrument to accommodate steering wire(s), which might increase the diameter of the elongate medical instrument.

In addition, since probe 24 is coupled to elongate medical instrument 20 in situ within vasculature 22, the additional steerability is provided to elongate medical instrument 20 without the need for a larger diameter delivery catheter for elongate medical instrument 20 than would otherwise be needed.

Probe 24 and this method also allow the healthcare worker to configure the resulting steering properties (e.g., bending point) of elongate medical instrument 20, such as by selecting the one or more locations along elongate medical instrument 20 at which distal longitudinal probe-portion 26 is coupled to distal longitudinal medical-instrument-portion 28. For example, steering properties of elongate medical instrument 20 may be configured based on the relative flexibility of different longitudinal segments of elongate medical instrument 20. Optionally, the healthcare worker may configure the steering properties based on medical imaging (e.g., a CT scan) and/or a planned vascular path of the particular procedure and the particular patient's anatomy.

For some applications, elongate medical instrument 20 is advanced through vasculature 22 along a first route 23A through vasculature 22, and probe 24 is advanced through vasculature 22 along a second route 23B through vasculature 22, second route 23B different from first route 23A.

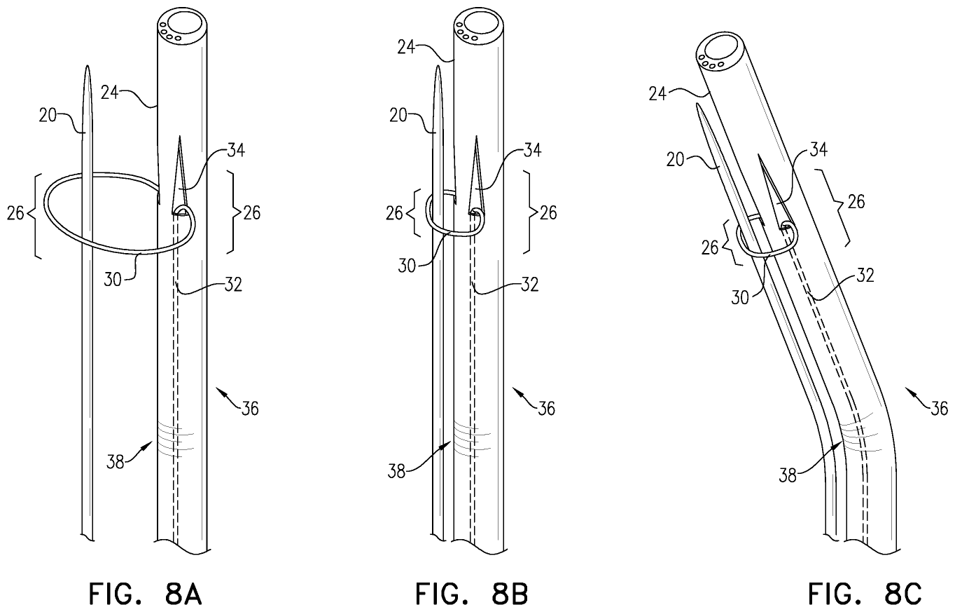

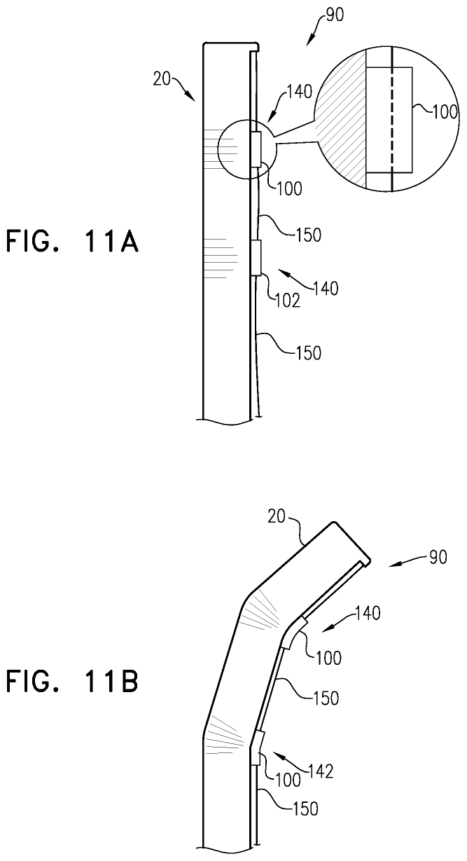

For some applications, probe 24 comprises a steerability-providing mechanism, which is configured to steer probe 24 so as to bend elongate medical instrument 20 within the body when distal longitudinal probe-portion 26 is mechanically coupled to distal longitudinal medical-instrument-portion 28. Probe 24 is used to bend elongate medical instrument 20 by activating the steerability-providing mechanism to steer distal longitudinal medical-instrument-portion 28 of probe 24. For example, the steerability-providing mechanism may comprise:
steerability-providing mechanism 36, described hereinbelow with reference to FIGS. 8A-C;
a steerability-providing mechanism similar to steerability-providing mechanism 36, described hereinbelow with reference to FIGS. 8A-C, other than that the steerability-providing mechanism comprises one or more dedicated steering wires, and optionally further comprises the one or more control wires 32, separate from the dedicated steering wires;
a steerability-providing mechanism 46, described hereinbelow with reference to FIGS. 10A-B; or
any steering mechanism known in the art, such as, by way of example and not limitation, the steering mechanisms described with reference to FIGS. 60-63D or FIGS. 64A-E of U.S. Pat. No. 7,666,204 to Thornton et al., which is incorporated herein by reference.

For some applications, such as shown in FIGS. 1A-B and 2A-E (and in FIGS. 3, 4A-B, 5A-B, 6A-B, 7A-B, 8A-C, 9A-E, and 10A-B, described hereinbelow), distal longitudinal probe-portion 26 comprises one or more loops 30, which are configured to be looped around distal longitudinal medical-instrument-portion 28 when distal longitudinal probe-portion 26 is positioned at least partially alongside distal longitudinal medical-instrument-portion 28 within the body, thereby mechanically coupling distal longitudinal probe-portion 26 to distal longitudinal medical-instrument-portion 28 within the body. Distal longitudinal probe-portion 26 is mechanically coupled to distal longitudinal medical-instrument-portion 28 by looping the one or more loops 30 around distal longitudinal medical-instrument-portion 28.

For some of these applications, looping the one or more loops 30 around distal longitudinal medical-instrument-portion 28 comprises passing the one or more loops 30 over the distal end of distal longitudinal medical-instrument-portion 28; and thereafter, proximally withdrawing distal longitudinal probe-portion 26 or distally advancing distal longitudinal medical-instrument-portion 28, such that the one or more loops 30 are positioned around distal longitudinal medical-instrument-portion 28.

For some of these applications, distal longitudinal probe-portion 26 is mechanically coupled to distal longitudinal medical-instrument-portion 28 by tightening the one or more loops 30 around distal longitudinal medical-instrument-portion 28 after looping the one or more loops 30 around distal longitudinal medical-instrument-portion 28. For example, probe 24 may comprise one or more control wires 32 that are configured to tighten the one or more loops 30, such as described hereinbelow with reference to FIGS. 4A-B, 5A-B, 6A-B, and 7A-B.

Reference is now made to FIG. 3, which is a schematic illustration of another configuration of probe 24, in accordance with an application of the present invention. The features of this configuration may be implemented in combination with any of the other features of probe 24 described herein, including with reference to FIGS. 1A-B and 2A-E. In this configuration, distal longitudinal probe-portion 26 includes two or more loops 30, and distal longitudinal probe-portion 26 is mechanically coupled to distal longitudinal medical-instrument-portion 28 by looping the two or more loops 30 around distal longitudinal medical-instrument-portion 28.

Reference is now made to FIGS. 4A-B, which are schematic illustrations of yet another configuration of probe 24, in accordance with an application of the present invention.

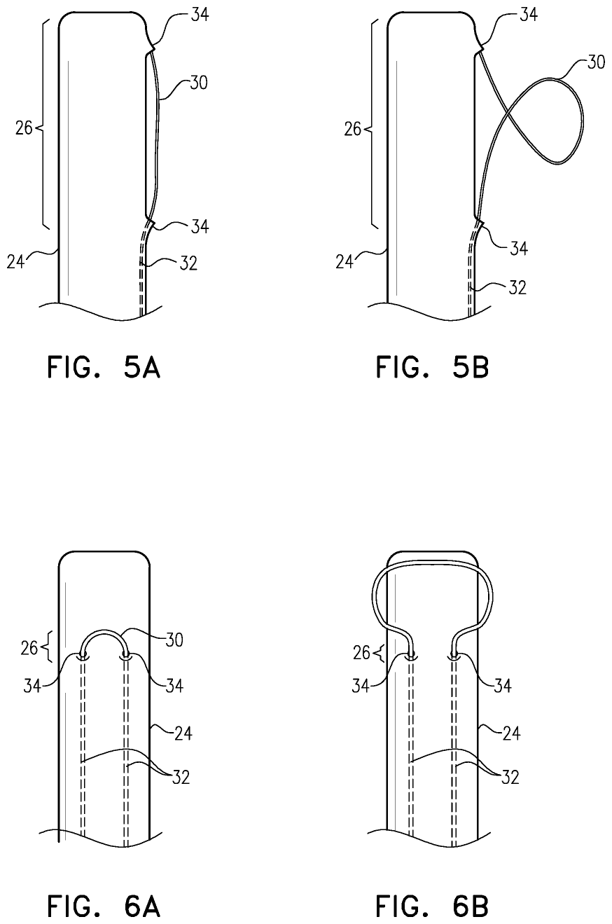

Reference is further made to FIGS. 5A-B, which are schematic illustrations of still another configuration of probe 24, in accordance with an application of the present invention.

Reference is still further made to FIGS. 6A-B, which are schematic illustrations of another configuration of probe 24, in accordance with an application of the present invention.

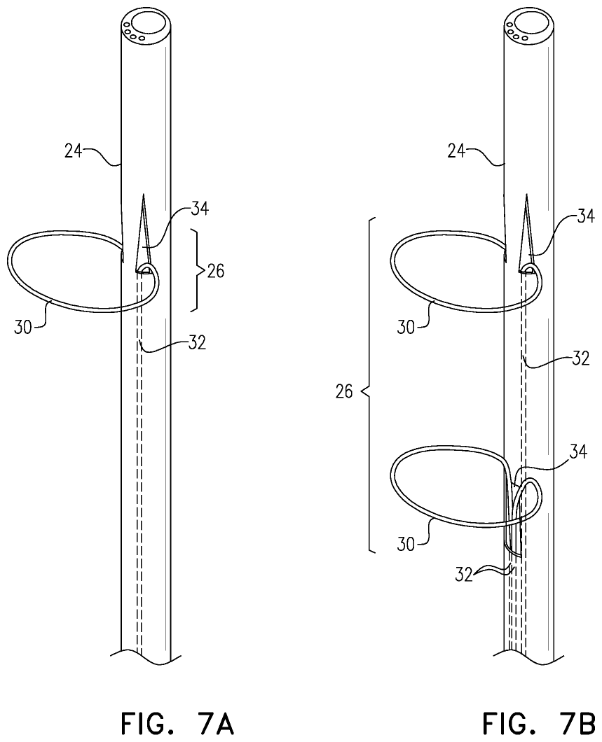

Reference is also made to FIGS. 7A-B, which are schematic illustrations of additional configurations of probe 24, in accordance with respective applications of the present invention.

The configurations shown in FIGS. 4A-B, 5A-B, 6A-B, and 7A-B may be implemented in combination with the configurations of probe 24 shown in the other figures, including, but not limited to, FIGS. 1A-B, 2A-E, and 3, mutatis mutandis.

In the configurations shown in FIGS. 4A-B, 5A-B, 6A-B, and 7A-B, the one or more loops 30 are configured to assume a non-radially-extended configuration by being retractable into and/or against probe 24 for advancing probe 24 through vasculature 22, such as shown in FIGS. 4A, 5A, and 6A (and in FIG. 2A, described hereinabove). In order to loop the one or more loops 30 around distal longitudinal medical-instrument-portion 28, the one or more loops 30 are transitioned to a radially-extended configuration in which the one or more loops 30 are extended away from probe 24, such as shown in FIGS. 4B, 5B, 6B, and 7A-B (and in FIGS. 1A and 2B, described hereinabove).

Optionally, the one or more loops 30 are enlarged as they are transitioned to the radially-extended configuration by being extended away from probe 24.

Optionally, the same mechanism is used in opposite manners to enlarge the one or more loops 30 during deployment of the one or more loops 30 away from probe 24, and to subsequently tighten the one or more loops 30 around distal longitudinal medical-instrument-portion 28 after looping the one or more loops 30 around distal longitudinal medical-instrument-portion 28.

Optionally, probe 24 comprises one or more control wires 32 that pass along (e.g., inside) probe 24 for controlling the extension and/or tightening of the one or more loops 30. The one or more loops 30 extend from one or more distal portions of the one or more control wires 32. The one or more control wires 32 are arranged such that:
distal pushing of the one or more control wires 32 transitions the one or more loops 30 to the radially-extended configuration by extending the one or more loops 30, and
proximal pulling of the one or more control wires 32 tightens the one or more loops 30, either (a) around distal longitudinal medical-instrument-portion 28, when distal longitudinal probe-portion 26 is positioned at least partially alongside distal longitudinal medical-instrument-portion 28, or (b) against an external surface of probe 24 in order to transition the one or more loops 30 back to the non-radially-extended configuration after decoupling from elongate medical instrument 20 for removal from the body.

Optionally, the one or more control wires 32 are integrally formed with the one or more loops 30, i.e., the one or more loops 30 are defined by one or more distal portions of the one or more control wires 32. Alternatively, the one or more control wires 32 and the one or more loops 30 are formed separately and coupled together during manufacture.

Optionally, all or a portion of the one or more loops pass through one or more openings 34 in a wall of probe 24, e.g., a single opening 34 per loop 30, as shown in FIGS. 4A-B, or two openings 34 per loop 30, as shown in FIGS. 5A-B, 6A-B, 7A-B, and 8A-C. For example, the two openings 34 may be arranged at different respective longitudinal locations along probe 24, such as shown in FIGS. 5A-B and 7B, and/or at different respective radial locations around probe 24, such as shown in FIGS. 6A-B.

Reference is now made to FIGS. 8A-C, which are schematic illustrations of a configuration of probe 24 comprising a steerability-providing mechanism 36, in accordance with an application of the present invention. For some applications, steerability-providing mechanism 36 comprises the one or more control wires 32 that pass through one or more channels defined by a wall of probe 24. Steerability-providing mechanism 36 is configured such that proximal pulling of at least one of the one or more wires 32 bends probe 24, such as shown in FIG. 8C. Activating steerability-providing mechanism 36 comprises pulling the at least one of the one or more wires 32 proximally to bend probe 24, such as shown in FIG. 8C.

For some of these applications, distal longitudinal probe-portion 26 comprises the one or more loops 30, the one or more wires 32 are attached to or define the one or more loops 30, respectively, and the one or more loops 30 are configured to be looped around distal longitudinal medical-instrument-portion 28 so as to mechanically couple distal longitudinal probe-portion 26 to distal longitudinal medical-instrument-portion 28, such as shown in FIGS. 8A-B. Pulling the at least one of the one or more wires 32 first decreases the diameter of the one or more loops 30 until elongate medical instrument 20 is mechanically coupled to probe 24, and then, when the diameter of the one or more loops 30 cannot further be reduced due to the presence of elongate medical instrument 20, probe 24 bends at the level of a bending region 38. For example, bending region 38 may comprise a softer material than other portions of probe 24, or may comprise the same material as other portions of probe 24, but with one or more cuts (e.g., half circular cuts), such as shown in FIGS. 8A-C, which allow probe 24 to bend along them without kinking.

Reference is now made to FIGS. 9A-E, which are schematic illustrations of another configuration of probe 24, in accordance with an application of the present invention. This configuration may be implemented in combination with the configurations of probe 24 shown in FIGS. 1A-B, 2A-E, and/or 9A-B, mutatis mutandis. As in these other configurations, probe 24 comprises a steerability-providing mechanism, such as described herein.

In this configuration, probe 24 comprises one or more loops 30 (for example, exactly one loop 30, as shown) that are pivotable with respect to an external surface of probe 24, for example about one or more respective shafts 54. Typically, the one or more loops 30 are pivotable between:
a non-radially-extended configuration, such as shown in FIG. 9A, in which the one or more loops 30 are in a collapsed configuration in which the one or more loops 30 are oriented parallel to a longitudinal axis of probe 24; typically, the one or more loops 30 are flush with the external surface of probe 24 when in the collapsed configuration; optionally, the external surface of probe 24 is shaped so as to define indentations 52, which are shaped so as to accept the one or more loops 30 when in the collapsed configuration, and
a radially-extended configuration, such as shown in FIGS. 9B-E, in which the one or more loops 30 extend radially outwardly from the external surface of probe 24, such as perpendicular to the external surface.

For some applications, probe 24 comprises one or more control wires 56, which are configured to transition the one or more loops 30 between the non-radially-extended configuration (collapsed configuration) and the radially-extended configuration and vice versa, such as by pivoting the one or more loops 30 about the one or more respective shafts 54, optionally using respective levers. More particularly, the one or more control wires 56 are arranged such that:
distal pushing of the one or more control wires 56 transitions the one or more loops 30 to the radially-extended configuration by pivoting the one or more loops 30 away from the external surface of probe 24, and
proximal pulling of the one or more control wires 56 transitions the one or more loops 30 to non-radially-extended configuration by pivoting the one or more loops 30 toward the external surface of probe 24.

For some applications, instead of, or in addition to, the one or more loops 30, probe 24 comprises one or more C-shaped clips 50, which are configured to snap onto distal longitudinal medical-instrument-portion 28 of elongate medical instrument 20. The one or more C-shaped clips 50 may be configured to assume non-radially-expanded (collapsed) and radially-expanded configurations as described hereinabove for the one or more loops 30.

For some applications, separate control wires 56 are provided for each of the one or more loops 30 and/or the one or more C-shaped clips 50 (configuration not shown). Alternatively, a same control wire 56 is provided for more than one loop 30 and/or more than one C-shaped clip 50, such as shown.

In some applications of the present invention, for example those described hereinabove with reference to FIGS. 1A-B, 2A-E, 3, and/or 9A-B, probe 24 comprises one or more C-shaped clips 50 instead of or in addition to the one or more loops 30.

Reference is now made to FIGS. 10A-B, which are schematic illustrations of a configuration of probe 24 comprising a steerability-providing mechanism 46, in accordance with an application of the present invention. steerability-providing mechanism 46 may be implement in combination with any of the configurations of probe 24 described herein, mutatis mutandis.

For some applications, steerability-providing mechanism 46 comprises a plurality of nested pre-shaped flexible catheters 48. At least a distal portion of each of catheters 48 is configured to assume a pre-defined shape when not constrained, including not constrained by one or more more-radially-outward catheters 48, a delivery sheath (not shown), anatomy of the subject, or otherwise. Catheters 48 are configured such that the overall shape of a given longitudinal segment is defined by the shape of the outermost catheter 48 along the segment. For example, the nested catheters 48 may have differing respective stiffnesses, with the more outer catheters having greater stiffnesses. For example, the relative stiffnesses may be due to the relative thicknesses or material properties of the catheters. The shapes of the longitudinal segments, and thus the overall shape of the probe 24, is set by controlling (a) the distance by which each catheter 48 is exposed from the radially closest more-radially-outward catheter 48 and (b) the rotation of the catheters 48 with respect to one another about the longitudinal axis. Steerability-providing mechanism 46 thus allows various shapes, orientations, and degrees of freedom.

Any of catheters 48 may comprise the one or more loops 30 described herein. For example, in FIG. 10B, the innermost catheter 48 is shown as comprising a single loop 30.

Reference is made to FIGS. 1A-10B. For some applications, a healthcare worker advances elongate medical instrument 20 through vasculature 22 and attempts to position elongate medical instrument 20 at a desired location within vasculature 22 without using probe 24. Probe 24 is advanced into vasculature 22 and coupled to medical instrument 20 only if the healthcare worker is not able to position elongate medical instrument 20 without use of probe 24.

Reference is still made to FIGS. 1A-10B. For some applications, probe 24 is axially non-compressible.

For some applications, probe 24 has an outer diameter of 2-8 mm.

Reference is still made to FIGS. 1A-10B. Optionally, the method may comprise advancing two or more probes 24, coupling them to elongate medical instrument 20, and using them to bend elongate medical instrument 20.

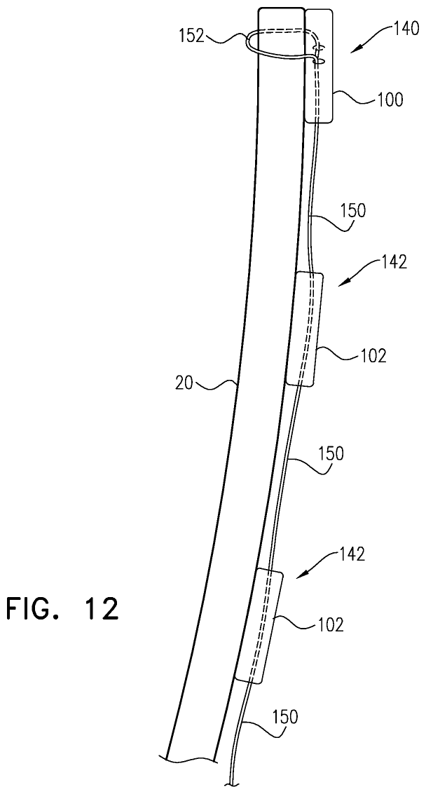

Reference is now made to FIGS. 11A-B, which are schematic illustrations of an attachable bending system 90 and a method of using attachable bending system 90 to intravascularly bend elongate medical instrument 20, in accordance with some applications of the present invention. Reference is also made to FIG. 12, which is a schematic illustration of a portion of attachable bending system 90, in accordance with some applications of the present invention.

As shown in FIGS. 11A and 12, attachable bending system 90 comprises:
a distal-most connector 100, which is configured to be mechanically coupled to elongate medical instrument 20 at a distal location 140 along elongate medical instrument 20;
one or more proximal-more connectors 102, which are configured to be mechanically coupled to elongate medical instrument 20 at one or more proximal-more locations 142 along elongate medical instrument 20, respectively, the one or more proximal-more locations 142 more proximal than distal location 140; and
a wire 150, which is fixed to distal-most connector 100 and slidably coupled to the one or more proximal-more connectors 102.

As shown in FIG. 11B, distal-most connector 100, the one or more proximal-more connectors 102, and wire 150 are arranged such that proximal pulling of wire 150 bends elongate medical instrument 20 within the body of the subject when distal-most connector 100 and the one or more proximal-more connectors 102 are coupled to elongate medical instrument 20 and elongate medical instrument 20 is within vasculature of the body.

The method of using attachable bending system 90 comprises:
mechanically coupling distal-most connector 100 to elongate medical instrument 20 at distal location 140 along elongate medical instrument 20, such as shown in FIGS. 11A and 12;
mechanically coupling the one or more proximal-more connectors 102 to elongate medical instrument 20 at the one or more proximal-more locations 142 along elongate medical instrument 20, respectively, the one or more proximal-more locations 142 more proximal the distal location 140, such as shown in FIGS. 11A and 12;
thereafter, introducing elongate medical instrument 20 into vasculature 22 and advancing elongate medical instrument 20 through vasculature 22; and
thereafter, bending elongate medical instrument 20 by pulling proximally on wire 150 fixed to distal-most connector 100 and slidably coupled to the one or more proximal-more connectors 102, such as shown in FIG. 11B.

Attachable bending system 90 and this method provide steerability to any elongate medical instrument 20 that lacks a steerability-providing mechanism, or provides additional steerability to any elongate medical instrument 20 that has a steerability-providing mechanism, such as to in order to provide additional directionality and/or a smaller bend radius to the existing steerability-providing mechanism. An alternative approach of integrating steerability functionality, or additional steerability functionality, directly into the elongate medical instrument during manufacture thereof would require adding additional channels to the elongate medical instrument to accommodate steering wire(s), which might increase the diameter of the elongate medical instrument.

In addition, since distal-most connector 100 and the one or more proximal-more connectors 102 are coupled to elongate medical instrument 20 before elongate medical instrument 20 is introduced into vasculature 22, practicing the method does not require additional vascular access than would otherwise be needed.

Attachable bending system 90 and this method also allow the healthcare worker to configure the resulting steering properties (e.g., bending point) of elongate medical instrument 20, such as by selecting distal location 140 and the one or more proximal-more locations 142 along elongate medical instrument 20 at which distal-most connector 100 and the one or more proximal-more connectors 102, respectively, are coupled to elongate medical instrument 20. For example, steering properties of elongate medical instrument 20 may be configured based on the relative flexibility of different longitudinal segments of elongate medical instrument 20. Optionally, the healthcare worker may configure the steering properties based on medical imaging (e.g., a CT scan) and/or a planned vascular path of the particular procedure and the particular patient's anatomy.

For some applications, the one or more proximal-more connectors 102 are shaped so as define one or more respective channels therethrough, and wire 150 passes through the one or more channels.

For some applications, distal-most connector 100 and the one or more proximal-more connectors 102 are mechanically coupled to elongate medical instrument 20 by gluing distal-most connector 100 and the one or more proximal-more connectors 102 to elongate medical instrument 20.

For some applications, such as shown in FIG. 12, distal-most connector 100 comprises a loop 152, and distal-most connector 100 is configured to be mechanically coupled to elongate medical instrument 20 by looping the loop around elongate medical instrument 20.

Reference is made to FIGS. 1A-12.

For some applications, such as shown in FIGS. 1A-B, elongate medical instrument 20 is an intracardiac medical instrument, and advancing elongate medical instrument 20 comprises advancing the intracardiac medical instrument through vasculature 22 into a heart chamber of the subject.

For some applications, elongate medical instrument 20 is a brain medical instrument, and advancing elongate medical instrument 20 comprises advancing the brain medical instrument through vasculature 22 into a brain of the subject.

For some applications, elongate medical instrument 20 is shaped so as to define at least one longitudinal channel therethrough. Alternatively, elongate medical instrument 20 is not shaped so as to define any longitudinal channels therethrough.

For some applications, elongate medical instrument 20 is a diagnostic medical instrument. For example, the diagnostic medical instrument may be selected from the group consisting of: an intracardiac echocardiography (ICE) probe, a biopsy probe, and an ultrasound probe.

For some applications, elongate medical instrument 20 is a therapeutic medical instrument. For example, the therapeutic medical instrument may be an ablation probe.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A probe for use with an elongate medical instrument, the probe configured to be advanced through vasculature of a body of a subject, the probe comprising:
a distal longitudinal probe-portion; and
one or more loops, which are disposed along the distal longitudinal probe-portion, and which are configured to be looped around a distal longitudinal medical-instrument-portion of the elongate medical instrument when the distal longitudinal probe-portion is positioned at least partially alongside a distal longitudinal medical-instrument-portion within the body, thereby mechanically coupling the distal longitudinal probe-portion to the distal longitudinal medical-instrument-portion within the body,
wherein the probe is configured to bend the elongate medical instrument within the body when the distal longitudinal probe-portion is mechanically coupled to the distal longitudinal medical-instrument-portion.

2. The probe according to claim 1, wherein the one or more loops are configured to be looped around the distal longitudinal medical-instrument-portion by passing the one or more loops over the distal end of the distal longitudinal medical-instrument-portion, and thereafter proximally withdrawing the distal longitudinal probe-portion or distally advancing the distal longitudinal medical-instrument-portion, such that the one or more loops are positioned around the distal longitudinal medical-instrument-portion.

3. The probe according to claim 1, wherein the distal longitudinal probe-portion comprises two or more loops, which are configured to be looped around the distal longitudinal medical-instrument-portion.

4. The probe according to claim 1, wherein the one or more loops are configured to be tightened around the distal longitudinal medical-instrument-portion after being looped around the distal longitudinal medical-instrument-portion, so as to mechanically couple the distal longitudinal probe-portion to the distal longitudinal medical-instrument-portion.

5. The probe according to claim 4, further comprising one or more control wires that pass along the probe, wherein the one or more loops extend from one or more distal portions of the one or more control wires, and are arranged such that proximal pulling of the one or more control wires tightens the one or more loops around the distal longitudinal medical-instrument-portion.

6. The probe according to claim 1, wherein the one or more loops are configured to assume a non-radially-extended configuration and a radially-extended configuration.

7. The probe according to claim 6, wherein the one or more loops are retracted into the probe when in the non-radially-extended configuration.

8. The probe according to claim 6, wherein the one or more loops are retracted against the probe when in the non-radially-extended configuration.

9. The probe according to claim 6, further comprising one or more control wires that pass along the probe and are configured to control a transition between the non-radially-extended configuration and the radially-extended configuration.

10. The probe according to claim 6, wherein the one or more loops are pivotable with respect to an external surface of the probe, so as to transition between the non-radially-extended configuration and the radially-extended configuration.

11. The probe according to claim 1, wherein the probe is axially non-compressible.

12. The probe according to any one of claims 1-11, further comprising a steerability-providing mechanism, which is configured to steer the probe so as to bend the elongate medical instrument within the body when the distal longitudinal probe-portion is mechanically coupled to the distal longitudinal medical-instrument-portion.

13. The probe according to claim 12, wherein the steerability-providing mechanism comprises one or more wires that pass through one or more channels defined by a wall of the probe, and the steerability-providing mechanism is configured such that proximal pulling of at least one of the one or more wires bends the probe.

14. The probe according to claim 13, wherein the one or more wires are attached to the one or more loops.

15. The probe according to claim 13, wherein the one or more wires are integral with the one or more loops.

16. A kit comprising the probe according to any one of claims 1-11, the kit further comprising the elongate medical instrument.

17. The kit according to claim 16, wherein the elongate medical instrument comprises an intracardiac medical instrument, which is configured to be advanced through the vasculature into a heart chamber of the subject.

18. The kit according to claim 16, wherein the elongate medical instrument comprises a brain medical instrument, which is configured to be advanced through the vasculature into a brain of the subject.

19. The kit according to claim 16, wherein the elongate medical instrument does not comprise a steerability-providing mechanism.

20. The kit according to claim 16, wherein the elongate medical instrument is shaped so as to define at least one longitudinal channel therethrough.

21. The kit according to claim 16, wherein the elongate medical instrument is not shaped so as to define any longitudinal channels therethrough.

22. The kit according to claim 16, wherein the elongate medical instrument comprises a diagnostic medical instrument.

23. The kit according to claim 22, wherein the diagnostic medical instrument is selected from the group consisting of: an intracardiac echocardiography (ICE) probe, a biopsy probe, and an ultrasound probe.

24. The kit according to claim 16, wherein the elongate medical instrument comprises a therapeutic medical instrument.

25. The kit according to claim 24, wherein the therapeutic medical instrument comprises an ablation probe.

26. An attachable bending system for use with an elongate medical instrument, the attachable bending system comprising:
a distal-most connector, which is configured to be mechanically coupled to the elongate medical instrument at a distal location along the elongate medical instrument;
one or more proximal-more connectors, which are configured to be mechanically coupled to the elongate medical instrument at one or more proximal-more locations along the elongate medical instrument, respectively, the one or more proximal-more locations more proximal than the distal location; and
a wire, which is fixed to the distal-most connector and slidably coupled to the one or more proximal-more connectors,
wherein the distal-most connector, the one or more proximal-more connectors, and the wire are arranged such that proximal pulling of the wire bends the elongate medical instrument within a body of a subject when the distal-most connector and the one or more proximal-more connectors are coupled to the elongate medical instrument and the elongate medical instrument is within vasculature of the body.

27. The attachable bending system according to claim 26, wherein the one or more proximal-more connectors are shaped so as define one or more respective channels therethrough, and wherein the wire passes through the one or more channels.

28. The attachable bending system according to claim 26, wherein the distal-most connector comprises a loop, and wherein the distal-most connector is configured to mechanically coupled to the elongate medical instrument by looping the loop around the elongate medical instrument.

29. A kit comprising the attachable bending system according to any one of claims 26-28, the kit further comprising the elongate medical instrument.

30. The kit according to claim 29, wherein the elongate medical instrument comprises an intracardiac medical instrument, which is configured to be advanced through the vasculature into a heart chamber of the subject.

31. The kit according to claim 29, wherein the elongate medical instrument comprises a brain medical instrument, which is configured to be advanced through the vasculature into a brain of the subject.

32. The kit according to claim 29, wherein the elongate medical instrument does not comprise a steerability-providing mechanism.

33. The kit according to claim 29, wherein the elongate medical instrument is shaped so as to define at least one longitudinal channel therethrough.

34. The kit according to claim 29, wherein the elongate medical instrument is not shaped so as to define any longitudinal channels therethrough.

35. The kit according to claim 29, wherein the elongate medical instrument comprises a diagnostic medical instrument.

36. The kit according to claim 35, wherein the diagnostic medical instrument is selected from the group consisting of: an intracardiac echocardiography (ICE) probe, a biopsy probe, and an ultrasound probe.

37. The kit according to claim 29, wherein the elongate medical instrument comprises a therapeutic medical instrument.

38. The kit according to claim 37, wherein the therapeutic medical instrument comprises an ablation probe.

39. A method comprising:
advancing an elongate medical instrument through vasculature of a body of a subject;
advancing a probe through the vasculature and positioning a distal longitudinal probe-portion of the probe at least partially alongside a distal longitudinal medical-instrument-portion of the elongate medical instrument;
thereafter, mechanically coupling the distal longitudinal probe-portion to the distal longitudinal medical-instrument-portion within the body; and
thereafter, using the probe to bend the elongate medical instrument within the body.

40. The method according to claim 39, wherein the distal longitudinal probe-portion includes one or more loops, and wherein mechanically coupling comprises looping the one or more loops around the distal longitudinal medical-instrument-portion.

41. The method according to claim 40, wherein looping the one or more loops around the distal longitudinal medical-instrument-portion comprises:
passing the one or more loops over the distal end of the distal longitudinal medical-instrument-portion; and
thereafter, proximally withdrawing the distal longitudinal probe-portion or distally advancing the distal longitudinal medical-instrument-portion, such that the one or more loops are positioned around the distal longitudinal medical-instrument-portion.

42. The method according to claim 40, wherein the distal longitudinal probe-portion includes two or more loops, and wherein mechanically coupling comprises looping the two or more loops around the distal longitudinal medical-instrument-portion.

43. The method according to claim 40, wherein mechanically coupling the distal longitudinal probe-portion to the distal longitudinal medical-instrument-portion comprises tightening the one or more loops around the one or more loops around the distal longitudinal medical-instrument-portion after looping the one or more loops around the distal longitudinal medical-instrument-portion.

44. The method according to claim 40,
wherein advancing the probe through the vasculature comprises advancing the probe through the vasculature while the one or more loops are in a non-radially-extended configuration, and
wherein mechanically coupling the distal longitudinal probe-portion to the distal longitudinal medical-instrument-portion comprises transitioning the one or more loops to a radially-extended configuration.

45. The method according to claim 44, wherein the one or more loops are retracted into the probe when in the non-radially-extended configuration.

46. The method according to claim 44, wherein the one or more loops are retracted against the probe when in the non-radially-extended configuration.

47. The method according to claim 44, wherein transitioning the one or more loops to the radially-extended configuration comprises using one or more control wires that pass along the probe to transition the one or more loops to the radially-extended configuration.

48. The method according to claim 44, wherein transitioning the one or more loops to the radially-extended configuration comprises pivoting the one or more loops with respect to an external surface of the probe.

49. The method according to claim 39, wherein the probe is axially non-compressible.

50. The method according to claim 39,
wherein advancing the elongate medical instrument through the vasculature comprises advancing the elongate medical instrument through the vasculature along a first route through the vasculature, and
wherein advancing the probe through the vasculature comprises advancing the probe through the vasculature along a second route through the vasculature, the second route different from the first route.

51. The method according to claim 39, wherein the probe includes a steerability-providing mechanism, and wherein using the probe to bend the elongate medical instrument comprises activating the steerability-providing mechanism to steer the probe.

52. The method according to claim 51, wherein the steerability-providing mechanism comprises one or more wires that pass through one or more channels defined by a wall of the probe, and wherein activating the steerability-providing mechanism comprises pulling at least one of the one or more wires proximally to bend the probe.

53. The method according to claim 52,
wherein the distal longitudinal probe-portion includes one or more loops,
wherein the one or more wires are attached to or integral with the one or more loops, and
wherein mechanically coupling comprises looping the one or more loops around the distal longitudinal medical-instrument-portion.

54. The method according to claim 39, wherein the elongate medical instrument is an intracardiac medical instrument, and wherein advancing the elongate medical instrument comprises advancing the intracardiac medical instrument through the vasculature into a heart chamber of the subject.

55. The method according to claim 39, wherein the elongate medical instrument is a brain medical instrument, and wherein advancing the elongate medical instrument comprises advancing the brain medical instrument through the vasculature into a brain of the subject.

56. The method according to claim 39, wherein the elongate medical instrument does not comprise a steerability-providing mechanism.

57. The method according to claim 39, wherein the elongate medical instrument is shaped so as to define at least one longitudinal channel therethrough.

58. The method according to claim 39, wherein the elongate medical instrument is not shaped so as to define any longitudinal channels therethrough.

59. The method according to claim 39, wherein the elongate medical instrument is a diagnostic medical instrument.

60. The method according to claim 59, wherein the diagnostic medical instrument is selected from the group consisting of: an intracardiac echocardiography (ICE) probe, a biopsy probe, and an ultrasound probe.

61. The method according to claim 39, wherein the elongate medical instrument is a therapeutic medical instrument.

62. The method according to claim 61, wherein the therapeutic medical instrument is an ablation probe.

63. A method comprising:
mechanically coupling a distal-most connector to an elongate medical instrument at a distal location along the elongate medical instrument;
mechanically coupling one or more proximal-more connectors to the elongate medical instrument at one or more proximal-more locations along the elongate medical instrument, respectively, the one or more proximal-more locations more proximal than the distal location;
thereafter, introducing the elongate medical instrument into vasculature of a body of a subject and advancing the elongate medical instrument through the vasculature; and
thereafter, bending the elongate medical instrument within the body by pulling proximally on a wire fixed to the distal-most connector and slidably coupled to the one or more proximal-more connectors.

64. The method according to claim 63, wherein the one or more proximal-more connectors are shaped so as define one or more respective channels therethrough, and wherein the wire passes through the one or more channels.

65. The method according to claim 63, wherein mechanically coupling the distal-most connector and the one or more proximal-more connectors to the elongate medical instrument comprises gluing the distal-most connector and the one or more proximal-more connectors to the elongate medical instrument.

66. The method according to claim 63, wherein the distal-most connector includes a loop, and wherein mechanically coupling the distal-most connector to the elongate medical instrument comprises looping the loop around the elongate medical instrument.

67. The method according to claim 63, wherein the elongate medical instrument is an intracardiac medical instrument, and wherein advancing the elongate medical instrument comprises advancing the intracardiac medical instrument through the vasculature into a heart chamber of the subject.

68. The method according to claim 63, wherein the elongate medical instrument is a brain medical instrument, and wherein advancing the elongate medical instrument comprises advancing the brain medical instrument through the vasculature into a brain of the subject.

69. The method according to claim 63, wherein the elongate medical instrument does not comprise a steerability-providing mechanism.

70. The method according to claim 63, wherein the elongate medical instrument is shaped so as to define at least one longitudinal channel therethrough.

71. The method according to claim 63, wherein the elongate medical instrument is not shaped so as to define any longitudinal channels therethrough.

72. The method according to claim 63, wherein the elongate medical instrument is a diagnostic medical instrument.

73. The method according to claim 72, wherein the diagnostic medical instrument is selected from the group consisting of: an intracardiac echocardiography (ICE) probe, a biopsy probe, and an ultrasound probe.

74. The method according to claim 63, wherein the elongate medical instrument is a therapeutic medical instrument.

75. The method according to claim 74, wherein the therapeutic medical instrument is an ablation probe.
